(19) United States
(12) Patent Application Publication
(10) Pub. No.: US 2023/0356199 A1
(43) Pub. Date: Nov. 9, 2023

(54) COMPOSITE CATALYST PHYSICALLY MIXED WITH NICKEL OXIDE AND METHOD FOR MANUFACTURING THE SAME

(71) Applicant: SEOUL NATIONAL UNIVERSITY R&DB FOUNDATION, Seoul (KR)

(72) Inventors: Do Heui KIM, Seoul (KR); Kihun NAM, Seoul (KR); Hae Won RYU, Seoul (KR)

(73) Assignee: SEOUL NATIONAL UNIVERSITY R&DB FOUNDATION, Seoul (KR)

(21) Appl. No.: 18/002,871

(22) PCT Filed: May 12, 2022
(86) PCT No.: PCT/KR2022/006796

§ 371 Date: Dec. 22, 2022

(30) Foreign Application Priority Data
Oct. 15, 2021 (KR) 10-2021-0137307

(51) Int. Cl. B01J 23/88; B01J 21/08; B01J 29/46; C07C 2/76; B01J 6/00; C07C 15/04; B01J 37/02

(52) U.S. Cl. CPC B01J 23/88; B01J 21/08; B01J 29/46; C07C 2/76; B01J 6/001; C07C 15/04; B01J 37/0201; B01J 37/0236

(57) ABSTRACT

Provided is a composite catalyst used in a dehydro-aromatization reaction of methane, the composite catalyst including a glasslike metal oxide catalyst which includes a supported catalyst including a porous support and a catalyst of a transition metal oxide supported on the support, and a nickel oxide (NiO) physically dispersed in the supported catalyst.

### TECHNICAL FIELD

The present invention relates to a composite catalyst used in a dehydro-aromatization reaction of methane and a preparation method of the composite catalyst.

### BACKGROUND ART

Natural gas and shale gas containing methane as the main component are promising alternative carbon sources which can replace petroleum. While coal and petroleum produce molecules with a high content of nitrogen and sulfur, thereby causing environmental pollution, methane, the main component of natural gas and shale gas emits mainly carbon dioxide and water vapor during combustion, and thus, is a clean, environmentally friendly fossil fuel.

With the recent increase in the development of natural gas and shale gas, the supply of light hydrocarbons, especially methane, continues to increase. Nevertheless, BTX, which is currently produced industrially, is dependent on a naphtha reforming process based on crude oil. Therefore, the development of a catalyst for a direct dehydro-aromatization reaction using methane is of considerable process importance in that it can reduce the dependence of crude oil. As a current BTX production process, a Cyclar process, an Aromax process, and the like which produce BTX from a C₃/C₄ gas are commercialized, and a catalyst used therefor is a catalyst in which a metal such as gallium is supported in ZSM-5 zeolite, and a direct dehydro-aromatization process using methane alone is not in the commercial stage, and a catalyst being studied is largely similar to a catalyst in which an oxide of a metal such as molybdenum is supported in ZSM-5.

In the dehydro-aromatization reaction of methane, the molybdenum oxide present in the molybdenum/zeolite catalyst undergoes carburization and is converted into molybdenum carbide or a similar reduced state, wherein this converting process becomes a rate-determining step reaction in the dehydro-aromatization reaction of methane of the molybdenum/zeolite catalyst that activates methane, and thereafter, the formed intermediate undergoes cyclization, thereby producing BTX, and at this time, carbon deposition also occurs. Therefore, in order to suppress catalyst deactivation, there is a still a task of efficiently dispersing the molybdenum carbide formed during the dehydro-aromatization reaction of methane without agglomeration in the corresponding catalyst system.

### BRIEF DESCRIPTION OF THE INVENTION

### Technical Problem

An aspect of the present invention provides a composite catalyst having higher reaction activity and stability than a typical catalyst by physically mixing a nickel oxide with a metal-supported catalyst, and a preparation method of the composite catalyst.

### Technical Solution

According to an aspect of the present invention, there is provided a composite catalyst used in a dehydro-aromatization reaction of methane, the composite catalyst including a glasslike metal oxide catalyst which includes a supported catalyst including a porous support and a catalyst of a transition metal oxide supported in the support, and a nickel oxide (NiO) physically dispersed in the supported catalyst.

According to another aspect of the present invention, there is provided a method for preparing a composite catalyst, the method including impregnating a porous support with a transition metal-containing precursor solution, calcining the support to prepare a supported catalyst in which a catalyst of the transition metal oxide is supported, and physically dispersing a nickel oxide in the supported catalyst.

### Advantageous Effects

According to a composite catalyst used in a dehydro-aromatization reaction of methane and a preparation method of the composite catalyst, by physically mixing a nickel oxide to a predetermined content, it is possible to exhibit higher catalytic activity and stability than those of a transition metal oxide supported catalyst and the like prepared by a typical supporting method. In addition, by using the composite catalyst of the present invention, there is an effect of increasing the BTX yield in a dehydro-aromatization reaction of methane compared to when a typical catalyst is used.

### BRIEF DESCRIPTION OF THE DRAWINGS

FIG. 1 is a view schematically showing a method for preparing a composite catalyst used in a dehydro-aromatization reaction of methane according to an embodiment of the present invention.

FIG. 2a and FIG. 2b are views showing a methane conversion and a BTX yield according to the molar ratio of Ni/Mo in a composite catalyst of Preparation Example 1 and a supported catalyst of Comparative Preparation Example 1.

FIG. 3a and FIG. 3b are views showing a methane conversion and a BTX yield according to the molar ratio of Ni/Mo in the supported catalyst of Comparative Preparation Example 1 and a supported catalyst of Comparative Preparation Example 2.

FIG. 4a to FIG. 4c are views showing the results of X-ray diffraction analysis before a reaction, after post-treatment, and after the reaction of the composite catalyst of Preparation Example 1 and the supported catalyst of Comparative Preparation Example 1.

FIG. 5a to FIG. 5e are views showing the results of a methane temperature programmed surface reaction(CH₄-TPSR) according to the molar ratio of Ni/Mo in the composite catalyst of Preparation Example 1, a nickel oxide, and the supported catalyst according to Comparative Preparation Example 1.

FIG. 6a to FIG. 6f are views showing TEM images and the degree of diameter dispersion of MoCx and cokes according to the molar ratio of Ni/Mo in the composite catalyst of Preparation Example 1 and the supported catalyst of Comparative Preparation Example 1.

FIGS. 7a and 7b are views showing the results of nitrogen adsorption/desorption analysis after post-treatment and after a reaction of the composite catalyst of Preparation Example 1 and the supported catalyst according to Comparative Preparation Example 1.

FIG. 8a and FIG. 8b are views showing a methane conversion and a BTX yield when composite catalysts of Preparation Example 2 and Preparation Example 3 are applied.

FIG. 9a and FIG. 9b are views respectively showing TEM images of the composite catalysts of Preparation Example 2 and Preparation Example 3.

FIG. 10a to FIG. 10c are views respectively showing the results of X-ray diffraction analysis before a reaction, after post-treatment, and after the reaction of the composite catalysts of Preparation Example 2 and the Preparation Example 3.

FIG. 11a and FIG. 11b are views showing the results of a methane temperature programmed surface reaction(CH₄-TPSR) for Preparation Examples 2 and 3.

FIG. 12a to FIG. 12d are views showing TEM images after post-treatment and after a reaction for Preparation Examples 2 and 3.

### DETAILED DESCRIPTION OF THE INVENTION

Hereinafter, with reference to the accompanying drawings, a composite catalyst used in a dehydro-aromatization reaction of methane and a preparation method of the composite catalyst according to the present invention will be described in detail so that those skilled in the art can easily carry out the present invention.

The composite catalyst used in a dehydro-aromatization reaction of methane is a composite catalyst including a glasslike metal oxide catalyst which includes a supported catalyst including a porous support and a catalyst of a transition metal oxide supported in the support, and a nickel oxide (NiO) physically dispersed(mixed) in the supported catalyst.

The support may be a microporous or mesoporous support, and specifically, may include an aluminosilicate-based molecular sieve, or an metal-organic framework(MOF). Preferably, the support may be a zeolite, more preferably, selected from the group consisting of HZSM-5, ZSM-5, zeolite A, zeolite X, zeolite Y, and mordenite, and most preferably ZSM-5.

The support may have pores inside, and the diameter of the pores may be from 5 to 7 Å. Particularly, since ZSM-5 zeolite has both a 2-dimensional pore structure and a pore diameter close to the kinetic diameter of a benzene molecule (about 6.0 Å), the dehydro-aromatization reaction of methane may be more efficiently performed.

The support may include silicon and aluminum, and the activity of the catalyst may be controlled in accordance with the molar ratio of silicon to the aluminum (Si/Al₂). By controlling the Si/Al₂ ratio, it is possible to suppress the aggregation of a metal oxide present on the surface of the support, and by dispersing the metal oxide into micropores of the support, it is possible to promote the reaction.

The Si/Al₂ ratio in the support may be 15 to 30, preferably 20 to 25. When the Si/Al₂ ratio in the support satisfies the above numerical range, the dehydro-aromatization reaction is promoted, thereby increasing the productivity of BTX, but on the other hand, when the Si/Al₂ ratio in the support is less than the above range, the number of acid sites in the zeolite decreases, and when the Si/Al₂ ratio in the support is greater than the above range, the strength of acid sites is weakened, which may result in reducing the efficiency of the dehydro-aromatization reaction.

A transition metal in a transition metal oxide supported in the support may include any one selected from the group consisting of molybdenum (Mo), chromium (Cr), nickel (Ni), tungsten (W), palladium (Pd), ruthenium (Ru), gold (Au), rhenium (Re), rhodium (Rh), or a combination thereof, preferably molybdenum.

The molybdenum is a solid transition metal with a silvery white sheen, and hardly reacts with oxygen (O₂) or water at room temperature. The molybdenum starts to be oxidized by oxygen at 300° C. or higher, and may become a molybdenum trioxide (MoO₃) by high-temperature calcining such as a reaction with oxygen in the air at 600° C. The molybdenum trioxide (MoO₃) supported in a supported catalyst may be carburized by methane, which is a carbon source, and be converted into a molybdenum carbide (MoCx), and the molybdenum carbide (MoCx) may then convert the methane into benzene. Particularly, the dehydro-aromatization reaction of methane is determined by the activation rate of a C—H bond of methane, wherein a molybdenum carbide may activate the C—H bond of methane. Therefore, the rate at which the molybdenum oxide is converted into a molybdenum carbide may have a significant impact on the performance of a catalyst used in the dehydro-aromatization reaction of methane.

The molybdenum may be used in the form of a precursor when preparing a supported catalyst. Specifically, the molybdenum may be used in the form of nitrate, chloride, sulfur oxide, acetate or ammonium, and preferably, may be used in the form of ammonium.

On the external surface of the supported catalyst in which the catalyst of the transition metal oxide is supported, another transition metal oxide, for example, a molybdenum trioxide and the like may be distributed, and due to the aggregation of the another transition metal oxide, the activity of the supported catalyst or the composite catalyst used in a dehydro-aromatization reaction of methane including the supported catalyst may be reduced.

The supported catalyst may be one in which 1 to 20 wt % of a transition metal oxide, preferably 6 to 15 wt % of a transition metal oxide, is supported based on the total weight of the supported catalyst. If the content of the transition metal oxide is less than the above lower limit, the number of active sites decreases, and as a result, the effect of improving a dehydrogenation reaction during the dehydro-aromatization reaction of methane is insignificant, so that the conversion rate of methane, which is a reactant, and the yield of a product (an aromatic compound) may be reduced, and when the content of the transition metal oxide is greater than the above upper limit, the transition metal oxide agglomerates on the surface of the supported catalyst and on the agglomerated transition metal oxide, cokes are formed, thereby promoting catalyst deactivation, and as a result, there may be a problem in that the yield of the product no longer increases.

The composite catalyst used in the dehydro-aromatization reaction of methane may be in the form of a glasslike metal oxide catalyst by physically dispersing a nickel oxide in the supported catalyst. The nickel oxide may be at least one selected from the group consisting of NiO, Ni₂O₃, and NiO₂, preferably NiO.

When a composite catalyst in which the nickel oxide is physically dispersed in the supported catalyst is used, the nickel oxide may react with methane, which is a reactant, and reduced to a nickel metal species. The reduced nickel metal species may promote carburization of the transition metal oxide present on the surface of the support of the supported catalyst and convert the transition metal oxide into a form of a transition metal carbide. Accordingly, it is possible to suppress aggregation of transition metal oxides in the vicinity of a catalyst active site on the surface of the supported catalyst, and to improve the dispersion of the transition metal carbide on the surface of the catalyst, thereby improving the activity of the catalyst.

Particularly, compared to a composite catalyst in which a nickel metal species (i.e., a metallic nickel) is supported as in the prior art, when a composite catalyst in which a nickel oxide is physically dispersed in a supported catalyst as in the present invention is used, the catalyst performance is dramatically enhanced in the dehydro-aromatization reaction of methane, and although the cause thereof has not been clearly identified, the present inventors assume that the activity of a catalyst varies depending on whether or not the conversion of nickel into a transition metal carbide is inhibited by the formation of a composite with a transition metal oxide on the surface of the catalyst and the degree of inhibition. Specifically, as in the prior art, in the case of a composite catalyst obtained by simply impregnating a nickel metal species, the nickel metal species tends to form a composite more easily with a transition metal oxide present on the surface of a support, so that the conversion of the transition metal oxide into a carburized transition metal form (a transition metal carbide) is inhibited, and thus, most transition metal oxides are present without being carburized, which has been thought to lead to a problem in which the yield of a dehydro-aromatization reaction of methane is degraded, and the deactivation of the catalyst proceeds rapidly. However, in the case of a composite catalyst in which a nickel oxide is physically dispersed in a supported catalyst according to the present invention, unlike in the prior art, a transition metal oxide is converted into a carburized form (a transition metal oxide), and thus, is capable of participating in a dehydro-aromatization reaction of methane, which is thought to allow better catalyst performance to be exhibited.

The mechanism as described above, that is, a nickel oxide is reduced to a nickel metal species, and the reduced nickel metal species promotes the carburization of a transition metal oxide present on the surface of a support may be achieved though post-treatment of elevating the temperature from 400° C. to 700° C. In addition, the post-treatment process may be performed before the start of a dehydro-aromatization reaction of methane to increase the BTX yield of the methane in the reaction.

In addition, since the reduced nickel metal species induces the formation of cokes thereon to suppress carbon deposition on the active sites of supported catalyst, the activity of the composite catalyst for the dehydro-aromatization reaction may be improved.

The molar content of nickel derived from the nickel oxide in the composite catalyst may be 1 to 55 mol %, preferably 2 to 35 mol %, more preferably 2 to 25 mol %, and even more preferably 2 to 15 mol % based on the transition metal oxide. When the molar content of nickel is less than the lower limit, the activity and stability of the catalyst may be degraded, thereby decreasing the BTX yield, and when greater than the upper limit, a large amount of nickel metal species is formed from the nickel oxide to increase carbon deposits formed on the nickel species itself, which reduces the effect of promoting the reduction of a molybdenum oxide, so that the formation rate of active species may be degraded, and as a result, the BTX production may be degraded.

When the composite catalyst is used in the dehydro-aromatization reaction of methane, the reduction rate of the surface area of the composite catalyst before and after the reaction may be 20 to 25%, and the reduction rate of the micropore volume of the composite catalyst may be 20 to 30%. As a result of suppressing carbon deposition and blockage of pores on a supported catalyst due to the addition of a nickel oxide, the reduction rates of the catalyst surface area and micropore volume may become lower than that of the supported catalyst before the nickel oxide is added.

A method for preparing a composite catalyst used in a dehydro-aromatization reaction of methane according to another aspect of the present invention will be described in detail.

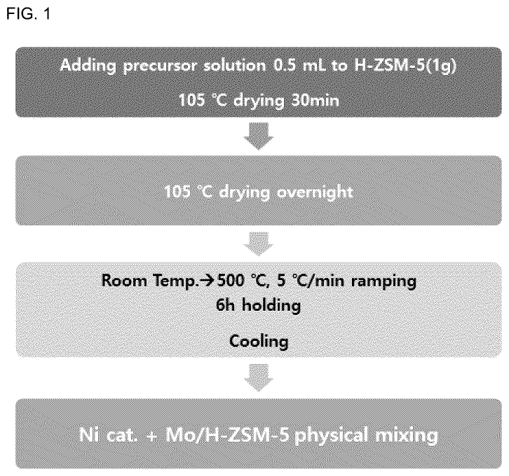

FIG. 1 schematically shows a method for preparing a composite catalyst used in a dehydro-aromatization reaction of methane of the present invention.

According to FIG. 1, the method may include impregnating a porous support with a transition metal-containing precusor solution, calcining the support to prepare a supported catalyst in which a catalyst of the transition metal oxide is supported, and physically dispersing a nickel oxide in the supported catalyst.

In the impregnation step, the transition metal-containing precursor solution may generally be one or more selected from nitrate-based, chloride-based, carbonate-based, and acetate-based, preferably a nitrate-based, and a solvent used may be one or more selected from water, ethanol, 1-propanol, 2-propanol, 1-butanol, 1-heptanol, and 1-hexanol, and preferably water.

In addition, the impregnation may be performed by a typical method such as liquid impregnation, vapor impregnation method, and a combination thereof, but a method thereof is not particularly limited.

The calcining of the support may be performed at 480 to 520° C. for 5 to 7 hours, preferably at 500° C. for 6 hours.

In the step of preparing a supported catalyst, after the calcining step, a step of drying the support may be further included. The drying of the support may be performed at 100 to 110° C. for 11 to 15 hours, preferably at 105° C. for 12 hours.

In the physical dispersion step, the nickel oxide may be physically mixed for 8 to 12 minutes. The mixing may be performed using a mortar, and the mixing method is not particularly limited.

Hereinafter, the present invention will be described in more detail with reference to examples. However, the following examples are merely illustrative of the present invention and are not intended to limit the scope of the present invention.

### EXAMPLES

### <Preparation Example 1> Preparation of Composite Catalyst in which Nickel Oxide is Physically Mixed

A molybdenum trioxide (MoO₃), an oxide of molybdenum, was supported in HZSM-5 zeolite by a typical impregnation method.

As a support, HZSM-5 was prepared and used by calcining NH₄-ZSM-5(Si/Al₂ ratio=23, Alfa Aesar Co., Ltd.) in a muffle furnace at 500° C. for 6 hours, and as a precursor of the oxide of molybdenum, a precursor solution was prepared and used by dissolving 1.0 g of ammonium heptamolybdate tetrahydrate (Sigma-Aldrich Co., Ltd.) in 8 mL of distilled water.

To 1 g of the prepared HZSM-5 zeolite, 0.5 m [symbol] of the precursor solution was added to impregnate the H-ZSM-5 zeolite such that the content of molybdenum was to be 12 wt %. Thereafter, drying was performed in an oven at 105° C. for 30 minutes, and the process was repeated 4 times in total. Lastly, a calcining process was performed, in which drying was performed in the oven at 105° C. for 12 hours or longer and then the temperature was maintained at 500° C. for 6 hours, to prepare a molybdenum oxide-supported zeolite catalyst (hereinafter, simply referred to as “Mo/HZSM-5”), which is a supported catalyst.

Thereafter, a nickel oxide (Sigma-Aldrich Co., Ltd.) was added to 1 g of Mo/HZSM-5 such that the molar ratio of Ni/Mo in the catalyst was respectively to be 5, 10, 20, 50, and 100%, and then physically mixed for 10 minutes using a mortar to prepare a composite catalyst (NiO—Mo/HZSM-5(PM)) which is used in a dehydro-aromatization reaction of methane.

According to the molar ratio of Ni/Mo, it was named xNiO—Mo/HZSM-5(PM). (x=Ni/Mo %, e.g., when Ni/Mo=10%, 10NiO—Mo/HZSM-5(PM)).

### <Preparation Example 2> Preparation of Composite Catalyst 1 in which Nickel Oxide Coated with Silica Layer is Physically Mixed

2.0 g of polyvinylpyrrolidone(PVP) was dissolved in 200 ml of ethanol, and then 0.4 g of a nickel oxide (Nickel(II) Oxide, green, −325 mesh, 99%, Sigma-Aldrich Co., Ltd.) was added thereto, followed by stirring the mixture for 12 hours. Thereafter, 25 mL of ammonia water (28%) was added to the mixture, and sonication was performed for 1 hour. Thereafter, 0.2 mL of tetraethylorthosilicate(TEOS) was added thereto dropwise, and centrifugation was performed. Thereafter, water and ethanol was mixed to perform washing, and then calcination was performed at 550° C. for 3 hours.

Thereafter, a molybdenum oxide-supported zeolite catalyst was prepared in the same manner as in Preparation Example 1, and the nickel oxide coated with a silica layer and the molybdenum oxide-supported zeolite were physically mixed using a mortar such that the ratio of Ni/Mo was to be 10 mol %, thereby preparing a composite catalyst (NiO@SiO₂).

### <Preparation Example 3> Preparation of Composite Catalyst 2 in which a Nano-Sized Nickel Oxide Coated with Silica Layer is Physically Mixed

A composite catalyst (nanoNiO@SiO₂) was prepared in the same manner as in Preparation Example 2 except that a commercially available nano-sized nickel oxide in a powder form (Nickel(II) Oxide, nanopowder, <50 nm particle size (TEM), 99.8% trace metals basis, Sigma-Aldrich Co., Ltd.) was used instead of the nickel oxide (Nickel(II) Oxide, green, −325 mesh, 99%, Sigma-Aldrich Co., Ltd.).

### <Comparative Preparation Example 1> Preparation of Supported Catalyst in which Nickel Oxide is Not Mixed

A molybdenum oxide-supported zeolite catalyst (Mo/HZSM-5), in which a nickel oxide is not mixed, was prepared as in the same manner as in Preparation Example 1.

### <Comparative Preparation Example 2> Preparation of Supported Catalyst in which Nickel is Additionally Supported

A molybdenum oxide-supported zeolite catalyst (Mo/HZSM-5) was prepared in the same manner as in Preparation Example 1, and by using nickel nitrate hexahydrate (Alfa Aesar Co., Ltd.) as a nickel precursor solution, 1 g of the prepared Mo/HZSM-5 catalyst was impregnated in 0.5 m [symbol] of the nickel precursor solution. Thereafter, drying was performed in an oven at 105° C. for 30 minutes, and the process was repeated 4 times in total. Lastly, a calcining process was performed, in which drying was performed in the oven at 105° C. for 12 hours or longer and then the temperature was maintained at 500° C. for 6 hours, to finally prepare a composite catalyst (Ni·Mo/HZSM-5(IMP)) in which nickel was additionally supported in addition to a molybdenum oxide.

According to the molar ratio of Ni/Mo, the corresponding catalyst was named yNi·Mo/HZSM-5(IMP). (y=Ni/Mo %, e.g., when Ni/Mo=10%, 10Ni·Mo/HZSM-5(IMP))

### <Example 1> BTX Production in Dehydro-Aromatization Reaction of Methane to which a Composite Catalyst in which Nickel Oxide is Physically Mixed is Applied

A dehydro-aromatization reaction of methane was performed in a quartz fixed bed reactor using the composite catalyst of Preparation Example 1 and the supported catalyst of Comparative Preparation Example 1.

Each of the catalysts was filled in a quartz fixed bed reactor having a diameter of 2 cm and a length of 50 cm, and then the catalyst was activated through a post-treatment process (activation treatment before use in a catalytic reaction). The temperature of the reactor was elevated at a rate of 20° C./min to 400° C. and 10° C./min to 700° C., and the temperature was maintained for 10 minutes to stabilize the temperature. The volume ratio of the post-treatment gas was CH₄:He:Ar=9:1:10, and the total gas flow rate was 20 m [symbol] /min. When the temperature was stabilized at 700° C., a gas having a composition of CH₄:He=9:1 was flowed into the reactor at a flow rate of 10 m [symbol] /min under atmospheric pressure to start the dehydro-aromatization reaction of methane.

The discharged gas was analyzed at regular intervals by on-line gas chromatography (YL6500GC) equipped with two columns (GS-Gaspro and Carboxen-1000) and two sensing devices (a flame ionization detector and a thermal conductivity detector). Argon gas was used as a carrier gas for the gas chromatography. Gas lines and gas valves at all reactor outlets were maintained at 230° C. to prevent condensation, and adsorption of a product such as naphthalene.

The conversion rate, selectivity, and yield of methane of carbon-containing products were calculated based on the mass balance of carbon using the He gas as the internal standard.

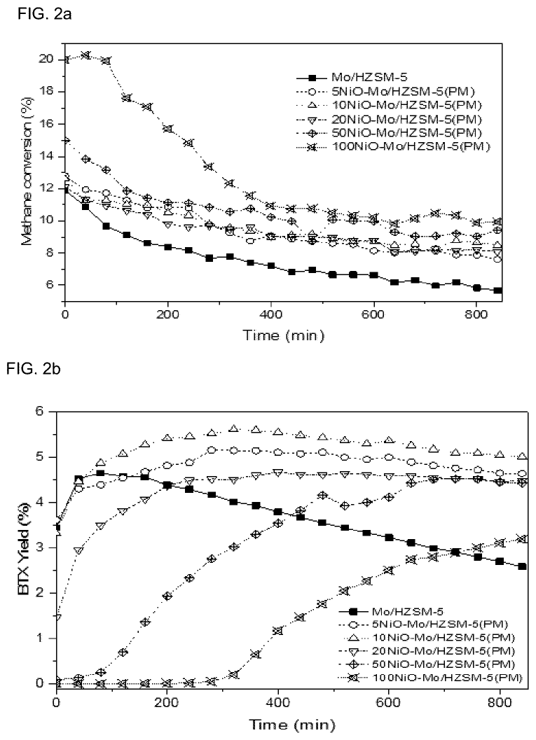

The methane conversion and BTX yield of the dehydro-aromatization reaction of methane in the case of using the composite catalyst of Preparation Example 1 and the supported catalyst of Comparative Preparation Example 1 are shown in FIGS. 2a and 2b.

Referring to FIG. 2a, it can be confirmed that in the case of a catalyst in which the nickel oxide according to Preparation Example 1 was physically mixed, the methane conversion, which is a reactant, increased regardless of the molar ratio of Ni/Mo when compared to that of Comparative Preparation Example 1 (wherein the nickel oxide was not mixed).

FIG. 2b shows the maximum BTX yield over time, and showed up to 5.6% when the molar ratio of Ni/Mo in the catalyst prepared in Preparation Example 1 was 10%. In addition, as the content of the nickel oxide increased, the deactivation rate was reduced. Particularly, 10NiO—Mo/HZSM-5(PM) showed a BTX yield of about 2 times higher than that of Comparative Preparation Example 1 (wherein a nickel oxide was not mixed) on the basis of a 14-hour long reaction, so that it was confirmed that by using a composite catalyst in which a nickel oxide was physically dispersed, the deactivation rate of the catalyst was decreased and the stability thereof was increased even after the dehydro-aromatization reaction of methane.

In addition, it is also possible to confirm an effect according to the molar ratio of Ni/Mo in the composite catalyst according to Preparation Example 1, which is that that both the methane conversion and the BTX yield increase when the molar ratio of Ni/Mo is 20% or lower, so that it can be inferred that the physically mixed nickel oxide plays an important role in activating methane and maintaining the activity of the methane during the reaction.

On the other hand, when the molar ratio of Ni/Mo in the catalyst is changed from 50% to 100%, the methane conversion increases from 15% to 20%, but thereafter sharply decreases and the BTX yield rate also decreases compared to when the molar ratio of Ni/Mo is 50%. In this case, it can be inferred that the nickel oxide converts methane to coke rather than BTX.

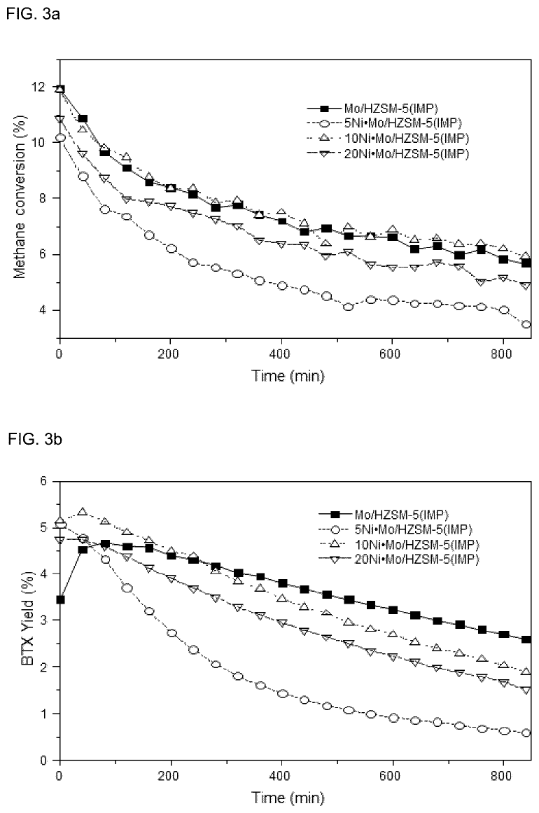

The methane conversion and BTX yield of the dehydro-aromatization reaction of methane of the supported catalyst prepared in Comparative Preparation Example 1 and the supported catalyst prepared in Comparative Preparation Example 2 are shown in FIGS. 3a and 3b.

In the case of the catalyst prepared according to Comparative Preparation Example 2 in which nickel was additionally supported, the conversion was lower than that of a catalyst to which a nickel oxide was not added (FIG. 3a) except when the molar ratio of Ni/Mo in the catalyst was 10%, and the maximum BTX yield was 5.3% when the molar ratio of Ni/Mo in the catalyst was 10%, but the BTX yield did not significantly increase (FIG. 3b). However, the catalyst in which nickel was additionally supported, even when the molar ratio of Ni/Mo in the catalyst was 10%, was deactivated faster than Comparative Preparation Example 1 (wherein a nickel oxide was not added) (FIG. 3b).

From the result, it can be confirmed that there is no great effect of improving catalyst performance when nickel is additionally supported, but the catalyst performance may be greatly improved through simple physically mixing of a nickel oxide. That is, it is assumed that when metallic nickel is supported, that is, impregnated with molybdenum (Comparative Preparation Example 2), the nickel forms a composite with the molybdenum, thereby playing a role of interfering with a process in which the molybdenum is converted into a carburized form of MoCx, so that the performance of the catalyst is degraded, whereas when a nickel oxide was physically mixed (Preparation Example 1), the above problem does not occur, thereby allowing the carburization process of the molybdenum to be smoothly performed, so that the performance of the catalyst is improved.

### <Example 2> XRD Analysis of Composite Catalyst in which Nickel Oxide is Physically Mixed

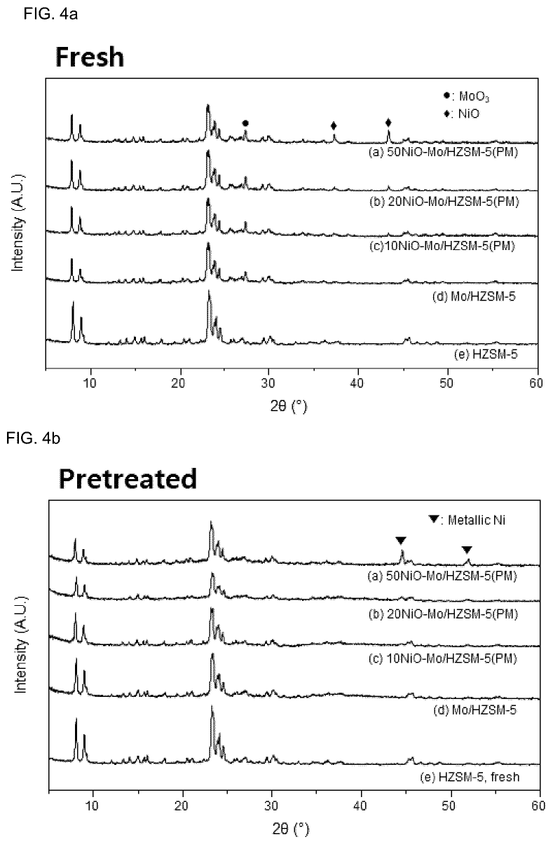

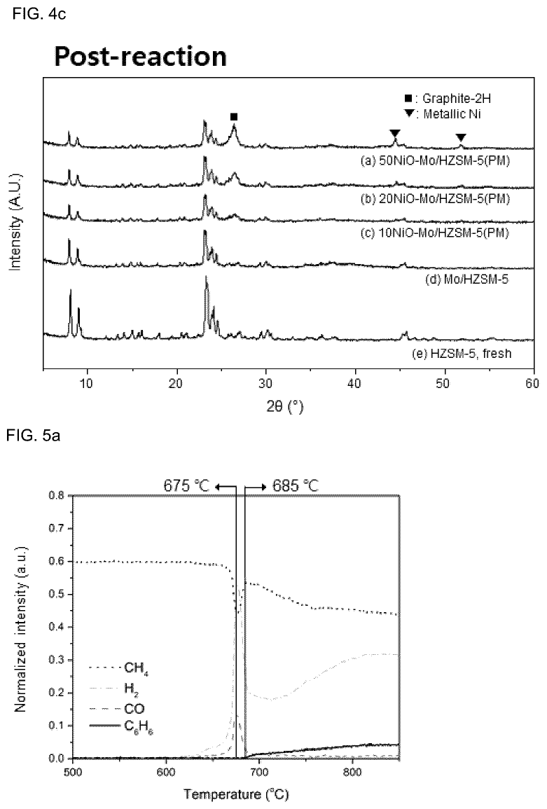

FIGS. 4a, 4b, and 4c respectively show XRD results of the composite catalyst according to Preparation Example 1, the supported catalyst according to Comparative Preparation Example 1, and a zeolite catalyst before the reaction(before the post-treatment), after the post-treatment, and 14 hours after the reaction.

Since a peak corresponding to HZSM-5 is identified in all catalysts, it can be seen that a basic crystal structure of zeolite is maintained even through the post-treatment of the composite catalyst, the dehydro-aromatization reaction of methane, and the like. A supported molybdenum oxide peak is identified in the XRD pattern for catalyst before the reaction(spectrum of (a) to (d)), which means that not all of the molybdenum oxide was efficiently dispersed on the surface of zeolite, even by the calcination step during the preparation process of the composite catalyst. In addition, no other nickel-related peaks other than the nickel oxide peak were observed.

Meanwhile, a peak related to molybdenum was not observed in the XRD patterns for catalyst after post-treatment (FIG. 4b). This means that through the post-treatment process, the molybdenum oxides present on the surface of the zeolite were efficiently dispersed on the surface of the zeolite as carburization (generation of MoCx) proceeded. In addition, a nickel metal peak is identified, which indicates that a nickel oxide is reduced to a nickel metal through the post-treatment process. Additionally, through the absence of a peak in which nickel and molybdenum are mixed, it can be seen that nickel and molybdenum do not form a composite during the post-treatment process.

Lastly, a Graphite-2H peak and a nickel metal peak are identified (FIG. 4c) in the XRD patterns for catalyst after the reaction. Through the absence of a peak in which molybdenum and nickel are mixed and the presence of the nickel metal peak, it can be seen that nickel continuously remains in the form of a nickel metal after the post-treatment. In addition, through the Graphite-2H peak, it can be confirmed that CNTs are formed in the catalyst.

### <Example 3> Analysis of temperature Programmed Surface Reaction of Methane(CH₄-TPSR) of Composite Catalyst in which Nickel Oxide is Physically Mixed

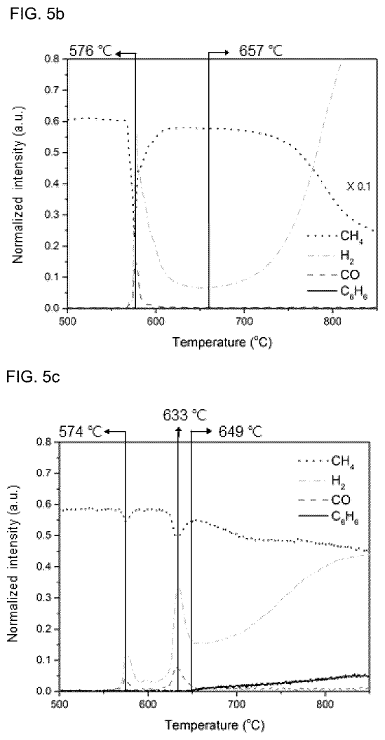

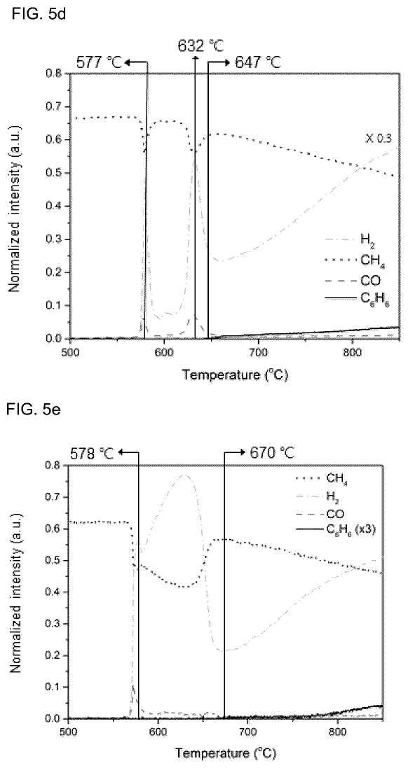

FIGS. 5a, 5b, 5c, 5d, and 5e show the results of a temperature programmed surface reaction of methane(CH₄-TPSR) in order to examine the effect of the post-treatment process on a supported catalyst, a nickel oxide, and a composite catalyst in which the nickel oxide is physically mixed.

First, FIG. 5a shows a H₂ peak and a CO peak at 675° C., as the result of the supported catalyst prepared according to Comparative Preparation Example 1. The peaks are shown since a molybdenum oxide (MoOx) is reduced to a molybdenum carbide (MoCx) by methane. The MoCx is an active state for benzene formation, and it can be confirmed that benzene is generated at 685° C.

FIG. 5b shows the CH₄-TPSR results of a nickel oxide (NiO), and it can be confirmed that NiO is reduced to a Ni metal species at 576° C. while simultaneously generating H₂ and CO. In addition, H₂ production increases again as the temperature is elevated to 657° C., which is due to the fact that the reduced Ni metal species converts methane to cokes and H₂. In addition, it can be seen that benzene is not generated from NiO even though methane is consumed.

FIG. 5c to FIG. 5e show the results for a composite catalyst (NiO—Mo/HZSM-5(PM) catalyst) in which a nickel oxide is physically mixed, wherein two peaks are shown each for the generation of H₂ and CO in all NiO—Mo/HZSM-5(PM) catalysts. The first peaks of H₂ and CO at 574° C. to 578° C. are due to methane decomposition during the reduction process of NiO to the Ni metal species, while the second peaks at a high temperature of 630° C. or higher are due to the reduction process of MoOx to MoCx. Particularly, when the molar ratio of Ni/Mo is 10 and 20, the second peaks of H₂ and CO due to the reduction of MoOx are shown respectively at 633° C. and 632° C., which is lower than the reduction temperature of MoOx by 40° C. compared to Comparative Preparation Example 1 (Mo/HZSM-5) in which a nickel oxide is not added. In addition, the benzene generation temperature, which is 649° C. when the molar ratio of Ni/Mo is 10 and 647° C. when the molar ratio of Ni/Mo is 20, is lower than the benzene generation temperature, which is 685° C., in the case of Mo/HZSM-5. That is, through the above results, it can be confirmed that the nickel oxide is reduced to the nickel metal species during the post-treatment process, and the reduced nickel metal species promotes the reduction of MoOx and the formation of MoCx, thereby promoting the generation of benzene. This is consistent with the XRD analysis results of Example 2.

The CH₄-TPSR results show that MoCx is formed at a relatively high temperature of 675° C. for Mo/HZSM-5, so that MoOx aggregates with higher mobility and blocks pores of zeolite before the reduction of MoOx, and as a result, the dispersion of MoCx decreases. On the other hand, in the case of the 10NiO—Mo/HZSM-5 catalyst, the Ni metal species promotes the reduction of MoOx at 633° C., which is 40° C. lower than the case of Mo/HZSM-5, and the lower reduction temperature reduces the mobility of MoOx, and as a result, the dispersion of MoCx is further improved.

In conclusion, in the post-treatment process, methane first reacts with the nickel oxide to form the Ni metal species, and then the Ni metal species promotes MoOx being reduced to MoCx and activated at a lower temperature than in the case of the supported catalyst, and as a result the activated MoCx promotes benzene generation.

In addition, through the comparison of 50NiO—Mo/HZSM-5(PM) and 10NiO—Mo/HZSM-5(PM), it can be confirmed that when a nickel oxide is added in more than an appropriate amount, a large amount of nickel metal species is formed and these nickel metal species increase carbon deposits formed by themselves, which reduces the effect of promoting the reduction of a molybdenum oxide, so that the formation active species is rather suppressed, and as a result, the production rate of benzene is slowed.

### <Example 4> TEM Image Analysis of Composite Catalyst in which Nickel Oxide is Physically Mixed

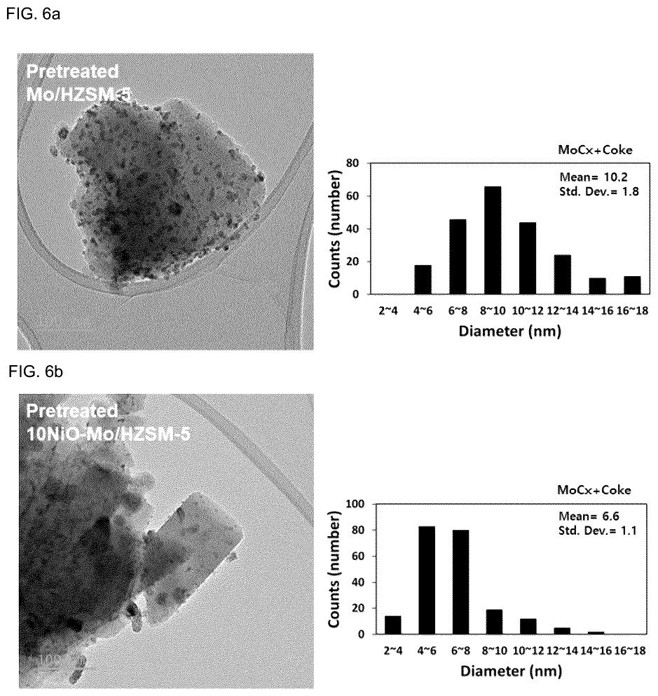

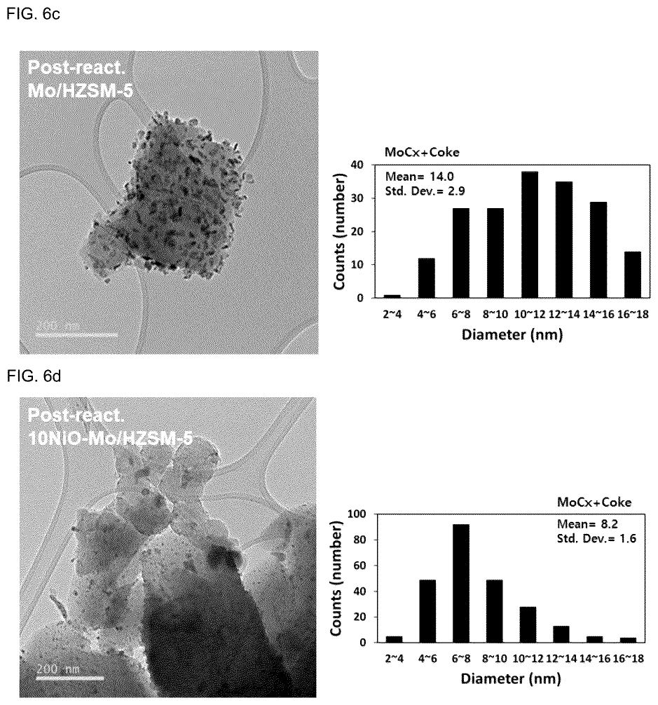

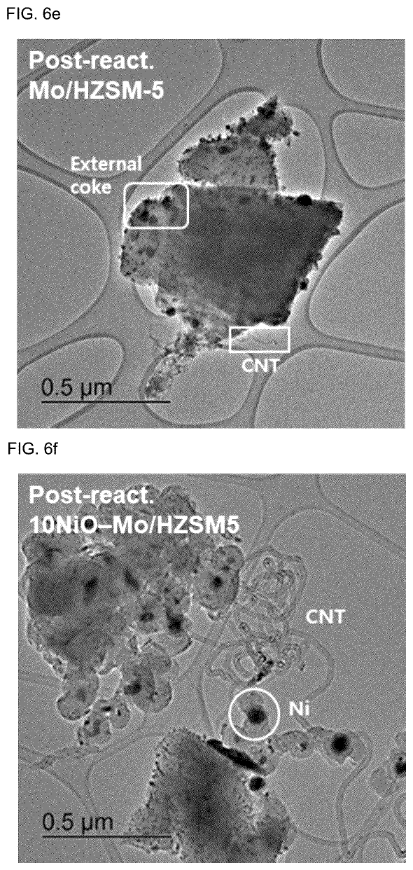

FIG. 6 shows TEM images of a composite catalyst and a supported catalyst. Through TEM, the size of particles with molybdenum carbide (MoCx) and cokes added thereto may be compared.

Regarding pretreated catalysts, in the case of Comparative Preparation Example 1 (Mo/HZSM-5) in which a nickel oxide is not mixed, the particles with MoCx and cokes added thereto has a diameter of 10.2 nm, whereas in the case of Preparation Example 1 in which a nickel oxide is physically mixed (molar ratio of Ni/Mo=10), the diameter of the particles is 6.6 nm, so that it can be confirmed that the dispersion of MoCx is higher.

Regarding post-reaction catalysts, in the case of Comparative Preparation Example 1 in which a nickel oxide is also not mixed, the diameter is 14.0 nm, and in the case of Preparation Example 1 in which a nickel oxide is physically mixed (molar ratio of Ni/Mo=10), the diameter is 8.2 nm, so that it can be confirmed that MoCx particles are much more efficiently dispersed.

In addition, in the case of Mo/HZSM-5, it can be confirmed that there are carbon deposits on the external surface of zeolite, whereas when the nickel oxide is physically mixed, it can be confirmed that carbon deposition occurs in the form of CNTs in the nickel metal species rather than on the surface of the zeolite.

In conclusion, through the TEM images, it can be confirmed that the dispersion of the molybdenum active species (MoCx) increases when the nickel oxide is physically mixed, the active species are less agglomerated, and there is less carbon deposition in the active species.

### <Example 5> Analysis of Surface Area and Pores of Composite Catalyst through Nitrogen Adsorption and Desorption Analysis

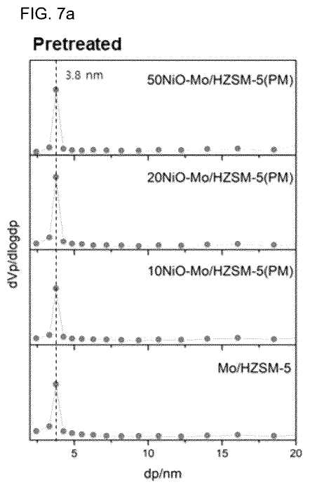

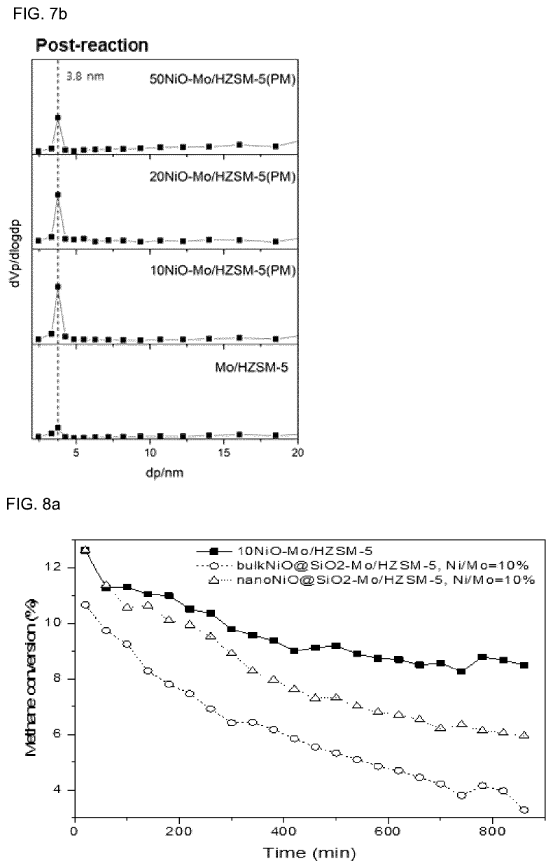

The catalysts of Preparation Example 1 and Comparative Preparation Example 1 having different Ni/Mo molar ratios were post-treated at a temperature of 150° C. and under vacuum conditions for 4 hours, and then physical adsorption of nitrogen was performed at −196° C. using ASAP 2010 (Micromeritics Co., Ltd.) equipment to obtain an adsorption/desorption isotherms, through which information on the BET surface area, micropore volume, and the like was obtained. The analysis results through nitrogen adsorption/desorption analysis are shown in Table 1 and FIGS. 7a and 7b below.

TABLE 1

| | | | | Surface | Micropore |
|---|---|---|---|---|---|
| | | BET | Micropore | area | volume |
| | | surface | volume | reduction | reduction |
| Catalyst | | area (m²/g) | (cm³/g) | rate (%) | rate (%) |
| Mo/ | After post- | 230 | 0.11 | 64.9 | 82.6 |
| HZSM-5 | treatment | | | | |
| | After | 81 | 0.02 | | |
| | reaction | | | | |
| 10NiO- | After post- | 220 | 0.09 | 23.4 | 21.0 |
| Mo/HZSM- | treatment | | | | |
| 5(PM) | After | 169 | 0.07 | | |
| | reaction | | | | |
| 20NiO- | After post- | 207 | 0.09 | 24.9 | 28.0 |
| Mo/HZSM- | treatment | | | | |
| 5(PM) | After | 155 | 0.06 | | |
| | reaction | | | | |
| 50NiO- | After post- | 160 | 0.06 | 28.4 | 19.8 |
| Mo/HZSM- | treatment | | | | |
| 5(PM) | After | 114 | 0.05 | | |
| | reaction | | | | |

In Table 1, it can be confirmed that compared to Comparative Preparation Example 1 in which a nickel oxide was not mixed, in the case of Preparation Example 1 in which a nickel oxide was physically mixed, the reduction in the catalyst surface area and micropore volume occurred less regardless of the Ni/Mo molar ratio. For example, looking at the 10NiO—Mo/HZSM-5(PM) catalyst which shows the best yield, Mo/HZSM-5 showed a reduction in catalyst surface area of 64.9% and a reduction in micropore volume of 82.6%, whereas 10NiO—Mo/HZSM-5(PM) has a reduction in catalyst surface area of 23.4% and a reduction in micropore volume of 21.0%, which are significantly lower.

Referring to FIGS. 7a and 7b, in the case in which a nickel oxide is not mixed (Comparative Preparation Example 1), pores of 3.8 nm are mostly blocked after the reaction compared to after the post-treatment, and thus, are not shown, but in the case in which a nickel oxide is physically mixed (Preparation Example 1), it can be confirmed that pores of the corresponding size are much more efficiently maintained even after the reaction. Through the above, it can be seen that the blockage of zeolite pores through carbon deposition is suppressed by physically mixing the nickel oxide.

### <Example 6> BTX Production in Dehydro-Aromatization Reaction of Methane when a Composite Catalyst in which Nickel Oxide Coated with Silica Layer is Physically Mixed is Applied

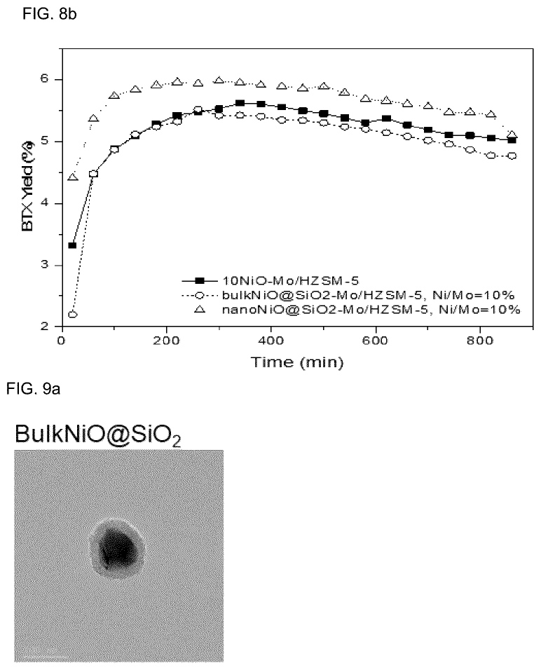

The dehydro-aromatization reaction of methane was performed in the quartz fixed bed reactor in the same manner as in Example 1 using the composite catalysts of Preparation Example 1, Preparation Example 2, and Preparation Example 3, respectively. The methane conversion and BTX yield of the dehydro-aromatization reaction of methane in the case of using the composite catalysts of Preparation Examples 2 and 3 are shown in FIGS. 8a and 8b.

When the catalyst of Preparation Example 2 (NiO@SiO₂) was used, the maximum BTX yield and deactivation rate were not significantly different compared to the case of using the catalyst of Preparation Example 1 (10NiO—Mo/HZSM-5(PM)). Meanwhile, when the catalyst of Preparation Example 3 (nanoNiO@SiO₂) was used, it was confirmed that the maximum BTX yield increased to 6.0%.

The corresponding embodiment shows that by physically mixing a nickel oxide with a modified surface, it is possible to enhance the activity and stability of a composite catalyst for a direct dehydro-aromatization reaction of methane, as well as to maximize the BTX yield, thereby showing the possibility of utilization through subsequent development.

### <Example 7> Properties Analysis and Comparison of Composite Catalysts in which Nickel Oxide Coated with Silica Layer is Physically Mixed

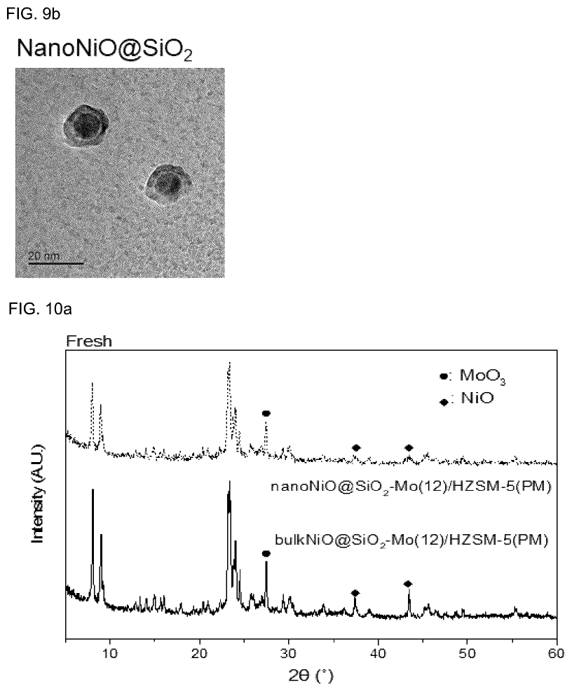

FIG. 9a and FIG. 9b show TEM images of the composite catalysts of Preparation Example 2 and Preparation Example 3. First, in the case of the catalyst of Preparation Example 2 (FIG. 9a) in which a commercially available nickel oxide is coated with a typical silica layer, the size was 100 to 120 nm, and the thickness of the silica layer was 32 nm. In the case of the catalyst of Preparation Example 3 (FIG. 9b) in which nano-sized nickel oxide is coated with a silica layer, the size of the nano-nickel oxide was 10 to 17 nm, and the thickness of the silica layer was 4 nm.

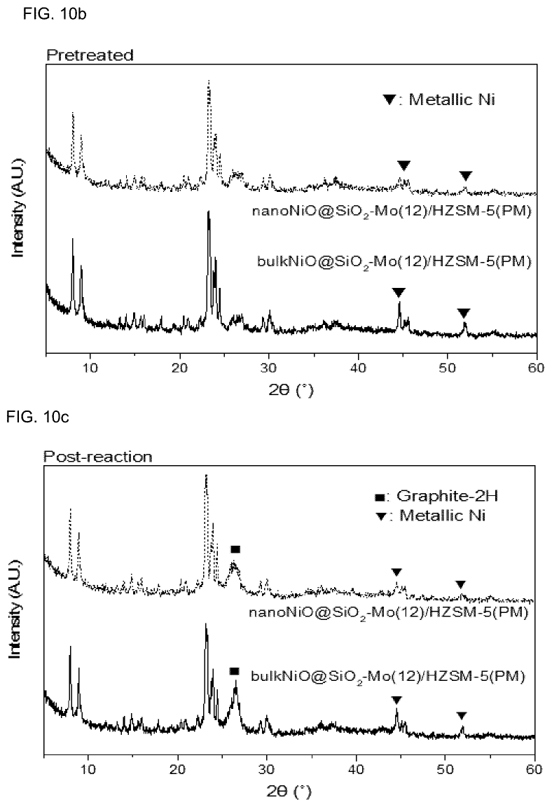

FIG. 10a to FIG. 10c show the results of X-ray diffraction analysis before a reaction (a), after post-treatment (b) and after the reaction (c) of the composite catalysts of Preparation Examples 2 and 3 in which the nickel oxide coated with the silica layer is physically mixed.

A peak of a molybdenum oxide can be seen in the composite catalyst (a) before the reaction, which means that not all of the molybdenum oxide is efficiently dispersed on the surface of the zeolite support even with calcination process. In addition, a peak related to the silica layer did not appear, no other nickel-related peaks other than the nickel oxide peak were observed.

Meanwhile, in the composite catalyst (b) after the post-treatment, a peak related to molybdenum was not observed. This means that through the post-treatment process, the molybdenum oxides present on the surface of the zeolite support were efficiently dispersed on the surface of the support as carburization proceeded. In addition, a nickel metal peak is identified, which means that the nickel oxide was reduced to a nickel metal through the post-treatment process. Additionally, through the absence of a peak in which nickel and molybdenum are mixed, it can be confirmed that nickel and molybdenum do not form a composite during the post-treatment process.

Lastly, in the composite catalyst (c) after the reaction, a Graphite-2H peak and a nickel metal peak were identified. Through the absence of a peak in which molybdenum and nickel are mixed and the presence of the nickel metal peak, it can be seen that nickel continuously remains in the form of a nickel metal through the reaction after the post-treatment. In addition, through the Graphite-2H peak, it can be confirmed that CNTs are formed in the catalyst.

The above results mean that even if a nickel oxide is coated with a silica layer, the reaction process is not changed thereby when compared to a case in which the silica layer is not applied.

### <Example 8> Analysis of Temperature Programmed Surface Reaction of Methane(CH₄-TPSR) of Composite Catalyst in which Nickel Oxide Coated with Silica Layer is Physically Mixed

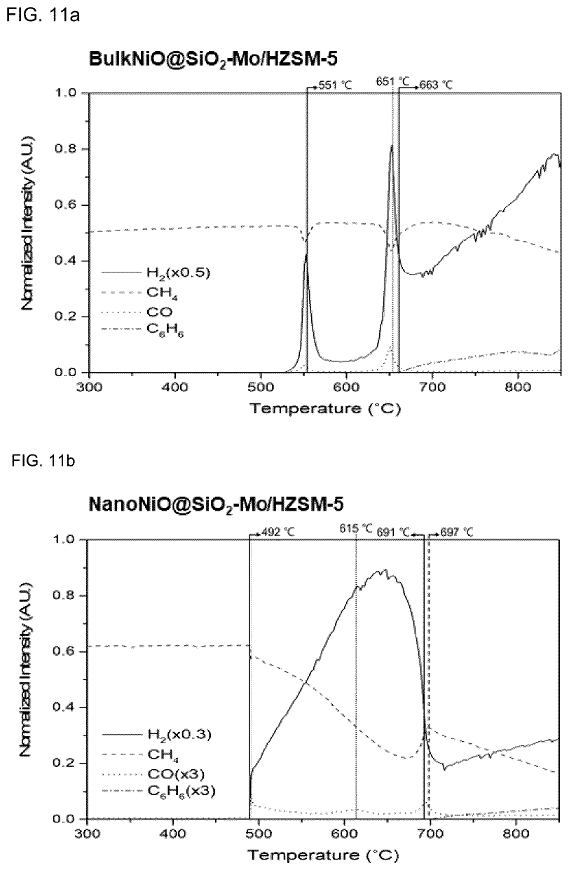

FIG. 11a and FIG. 11b show the results of a temperature programmed surface reaction of methane(CH₄-TPSR) of a composite catalyst of Preparation Examples 2 and 3 in which a nickel oxide coated with a silica layer is physically mixed.

In the case of Preparation Example 2 in which a nickel oxide is coated with a silica layer, it can be confirmed that a carburization peak of a molybdenum oxide was slightly delayed compared to the case of the 10NiO—Mo/HZSM-5 composite catalyst of Example 3 (FIG. 11a), which is presumably caused by the slow mass transfer of a methane-related active species by the silica layer.

As the size of the nickel oxide was reduced to a nano size, the reduction of the nickel oxide proceeded at a lower temperature. Thereafter, a peak related to hydrogen generation appears in a wide temperature range from 500° C. to 700° C., and through a carbon monoxide (CO) peak, it can be confirmed that the reduction of the molybdenum oxide occurs by being divided into 615° C. and 691° C. unlike other catalysts (FIG. 11b). The hydrogen peak over a wide temperature range is interpreted to appear as methane is decomposed by a nickel metal.

In conclusion, although carbon is deposited in the composite catalyst during the above process, the lowered carburization (reduction) temperature of the molybdenum oxide is interpreted to be a main cause of the increase in reactivity.

### <Example 9> TEM Image Analysis of Composite Catalyst in which Nickel Oxide Coated with Silica Layer is Physically Mixed

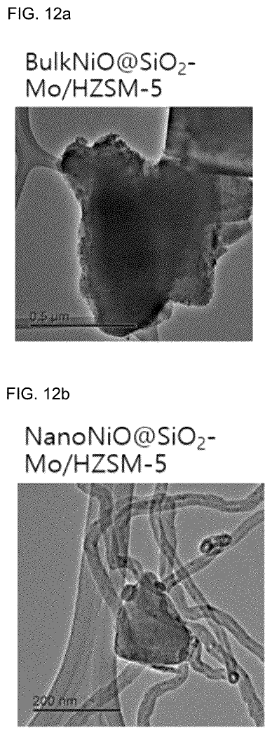

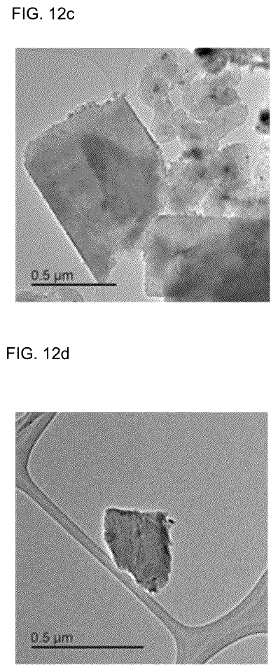

FIG. 12a to FIG. 12d show TEM images after the post-treatment and after the reaction of the composite catalysts (Preparation Examples 2 and 3) in which a nickel oxide coated with a silica layer is physically mixed.

First, through the TEM images of FIG. 12a and FIG. 12b, it is possible to compare sizes of combined particles of a molybdenum carbide and cokes for the composite catalysts of Preparation Examples 2 and 3 after the post-treatment. In the case of the catalyst using a commercially available nickel oxide (Preparation Example 2), the size was 7 nm (FIG. 12a), and in the case of the catalyst using a nano-sized nickel oxide(Preparation Example 3), the size was 5 nm (FIG. 12b), through which it can be indirectly confirmed that when the nano-sized nickel oxide was used, the dispersion of molybdenum was improved despite some occurrence of carbon deposition in the form of carbon nanotubes during the post-treatment process. In addition, the head portion of a CNT is empty, through which it can be confirmed that the reaction partially proceeded through the formation of carbon nanotubes while the size of a nickel metal species was maintained, and even though the silica layer was not maintained, there was no interference with a subsequent reaction due to the presence of nickel metal species.

Through the TEM images of FIG. 12c and FIG. 12d, it can be confirmed that in the composite catalysts of Preparation Examples 2 and 3 after the reaction, the size of an active sites of molybdenum and particles of carbon deposits formed at the corresponding point is 8.9 nm in the case of a catalyst using a commercially available nickel oxide (Preparation Example 2), and is 7.4 nm in the case of a catalyst in which a nano-sized nickel oxide was used (Preparation Example 3), so that the size is much smaller in the case of using the nano-sized nickel oxide. In addition, it is not possible to observe that the zeolite is affected, which shows that the molybdenum oxide supported zeolite is well maintained without the formation of a special composite of molybdenum and nickel.

1. A composite catalyst used in a dehydro-aromatization reaction of methane, the composite catalyst comprising a glasslike metal oxide catalyst which includes:
a supported catalyst including a porous support and a catalyst of a transition metal oxide supported in the support; and
a nickel oxide (NiO) physically dispersed in the supported catalyst.

2. The composite catalyst of claim 1, wherein the support comprises an aluminosilicate-based molecular sieve.

3. The composite catalyst of claim 1, wherein the support comprises zeolite.

4. The composite catalyst of claim 1, wherein the transition metal comprises at least one selected from the group consisting of molybdenum (Mo), chromium (Cr), nickel (Ni), tungsten (W), palladium (Pd), ruthenium (Ru), gold (Au), rhenium (Re), rhodium (Rh), or a combination thereof.

5. The composite catalyst of claim 1, wherein the supported catalyst contains 10 to 15 wt % of a transition metal oxide based on the total weight of the supported catalyst.

6. The composite catalyst of claim 1, wherein the molar content of nickel derived from a nickel oxide in the composite catalyst is 1 to 55 mol % with respect to the transition metal oxide.

7. The composite catalyst of claim 1, wherein the nickel oxide is included in a form dispersed in a silica support.

8. The composite catalyst of claim 1, wherein the BTX production yield of the dehydro-aromatization reaction of methane is 4.5 to 6%.

9. The composite catalyst of claim 1, wherein the reduction rate of the surface area of the composite catalyst before and after the dehydro-aromatization reaction of methane is 20 to 25%.

10. The composite catalyst of claim 1, wherein the reduction rate of the micropore volume of the composite catalyst before and after the dehydro-aromatization reaction of methane is 20 to 30%.

11. The composite catalyst of claim 1, wherein the support has an average diameter of 5 to 7 Å.

12. A method for preparing a composite catalyst, the method comprising:
impregnating a porous support with a transition metal-containing precursor solution;
calcining the support to prepare a supported catalyst in which a catalyst of the transition metal oxide is supported; and
physically dispersing a nickel oxide in the supported catalyst.

13. The method of claim 12, wherein the calcination of the support is performed at 480° C. to 520° C. for 5 to 7 hours.

14. The method of claim 12, further comprising, after the calcination of the support, performing drying.

15. The method of claim 14, wherein the drying is performed at 100 to 110° C. for 11 to 15 hours.
